(19) United States
(12) Patent Application Publication
(10) Pub. No.: US 2026/0355799 A1
(43) Pub. Date: Nov. 12, 2026

(54) Animal or Human Cremation Apparatus, And A Method of Cremating Animals or Humans

(71) Applicant: Drift Away Water Cremation Limited, BLAKEDOWN (GB)

(72) Inventor: Gregory Dickens, BLAKEDOWN (GB)

(73) Assignee: Drift Away Water Cremation Limited, BLAKEDOWN (GB)

(21) Appl. No.: 19/480,974

(22) PCT Filed: May 2, 2024
(86) PCT No.: PCT/GB2024/051156

§ 371 Date: Nov. 3, 2025

(30) Foreign Application Priority Data
May 3, 2023 (GB) 2306493.4

(51) Int. Cl. B09B 3/70; B09B 3/40

(52) U.S. Cl. CPC B09B 3/70; B09B 3/40

(57) ABSTRACT

The invention relates to an animal or human cremation apparatus, and a method of cremating animals or humans.

The animal or human water cremation apparatus comprising a fresh water supply 100, a first receptacle 30 designed to contain a liquid suitable for carrying out alkaline hydrolysis on an animal or human cadaver, and a second receptacle 70 designed to contain a liquid suitable for carrying out alkaline hydrolysis on an animal or human cadaver, and heating means 42 for heating liquid in the first receptacle and the second receptacle, and means for transferring hot waste liquid from the first receptacle to the second receptacle 70, or vice versa.

The receptacle 30, 70 is designed to heat the liquid to, and maintain the liquid at, a temperature of about 70 degrees to about 100 degrees, and is designed to maintain the gaseous liquid at a pressure of between 0.8 bar and 1.1 bar.

The invention relates to an animal or human cremation apparatus, and a method of cremating animals or humans. In particular, the animal or human cremation apparatus is designed to carry out alkaline hydrolysis on an animal or human cadaver.

International Patent Application Publication Number WO/2009/044204 discloses a human cremation apparatus having recirculating fluids from a decomposition unit. In particular, WO/2009/044204 discloses aiming recirculated fluids at the head of the cadaver in the decomposition unit. WO/2009/044204 also discloses recirculating fluids to a heat exchanger to heat water which is then subsequently returned to the decomposition unit. for processing the next cadaver.

An aim of the present invention is to provide an improved, or at least an alternative, animal or human cremation apparatus.

According to a first embodiment of the invention there is provided an animal or human cremation apparatus in accordance with Claim 1.

It should be appreciated by the reader that liquid can be moved from any receptacle to any other tank receptacle.

In that way, a cremation process in a second receptacle can be initiated whilst a receptacle is being cleaned after a cremation process has been conducted therein, thereby speeding up a series of cremations.

International Patent Application Publication Number WO/2009/044204 discloses heating the liquid to about 150 degrees, furthermore the pressure of the liquid is maintained at about 4 to 5 bar, so as to increase the boiling point of the liquid, such that the liquid is not boiling.

An aim of the present invention is to provide an improved, or at least an alternative, animal or human cremation apparatus.

According to a second embodiment of the invention there is provided an animal or human cremation apparatus in accordance with Claim 15.

There is a further problem associated with containing vapour generated as a side effect of heating treatment fluid above 100 degrees Celsius.

A pressure vessel such as that disclosed in WO/2009/044204 is expensive. Furthermore, a pressure vessel such as that disclosed in WO/2009/044204 has operational considerations from a safety point of view. The Applicant has devised a simpler and cheaper apparatus, and one with less safety concerns.

The Applicant's solution is to condense vapour back to its liquid form and return it to the tanks and to attach a negative pressure extract system to actively scavenge vapour and carry it away to the effluent tank or ultimately externally via the fan. This is achieved by placing an inline extract fan close to an external vent, running ducting that ultimately terminates inside the tanks. The final pipework is high temperature steam pipe which takes the form of a high level manifold and splits into individual feeds that approach the tanks from around 1.5 metres higher than the tanks. This means that any steam that condenses in the pipe as it cools will be fed by gravity directly back into the tank. Any remaining steam gets carried along the high temperature pipe and is fed into the effluent storage tank, where it condenses into liquid. The dry, hot air that remains is carried out of the building, via the fan. This solution is observed to work exceptionally well and maintains a slight negative atmospheric pressure in the tanks at all times, almost eliminating foaming, whilst allowing the natural (aggressive) bubbling of boiling liquid to act as a very effective fluid circulation mechanism that keeps liquid actively flowing over the biological remains to speed up decomposition. Using this system eliminates the requirement to have an active recirculation system, as detailed in International Patent Application Publication Number WO/2009/044204, saving cost and reducing complexity.

A different, separate, aim of the present invention is to provide an improved, or at least an alternative, methods of cremating animals or humans.

According to a third embodiment of the invention there is provided a method of cremating animals or humans in accordance with Claim 26.

Other optional and preferred features of an embodiment of the invention are set out in the dependent claims and/or the description, below.

It should be appreciated by the reader that the features of each of the independent claims can be combined in any complimentary manner with one or more features of another independent claim, and/or with any one or more of the dependent claims, and/or with any one or more features of the description, where such a combination of features would provide a working embodiment of the invention.

An animal or human cremation apparatus, and a method of cremating animals or humans in accordance with an embodiment of the invention will now be described, by way of example only, with reference to the accompanying drawings, in which,

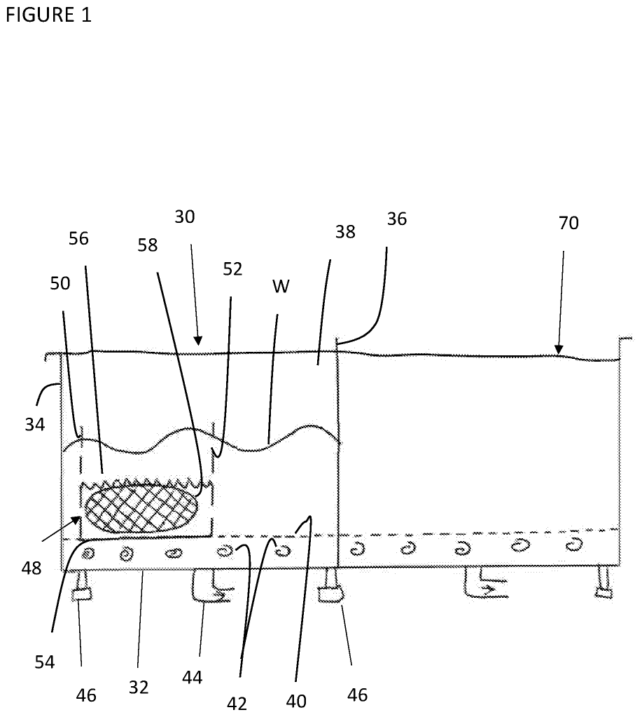

FIG. 1 is a schematic side view, in cutaway, of part of an animal or human cremation apparatus in accordance with an embodiment of the invention, in particular a plurality of tanks,

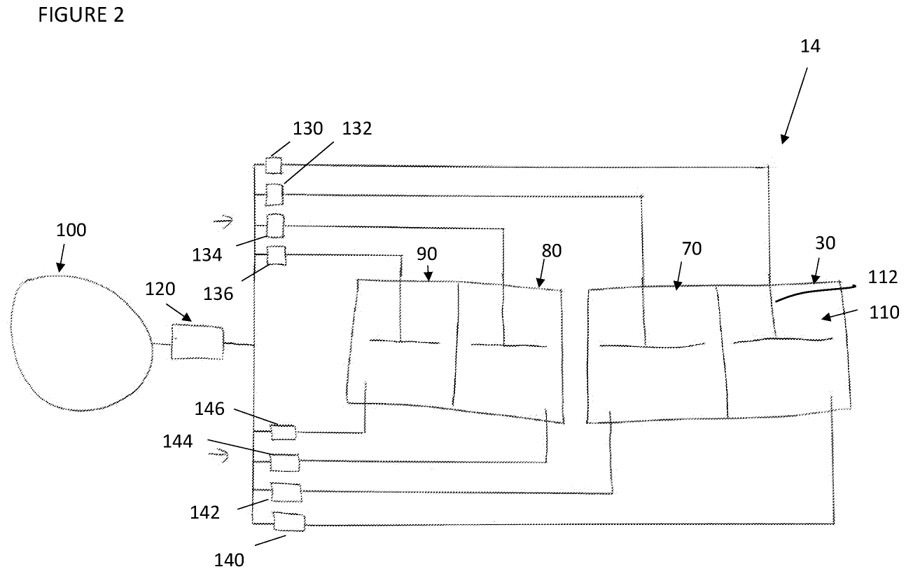

FIG. 2 is a schematic plan view of another part of the animal or human cremation apparatus of FIG. 1, in particular a plurality of tanks arranged in liquid communication with a fresh water supply,

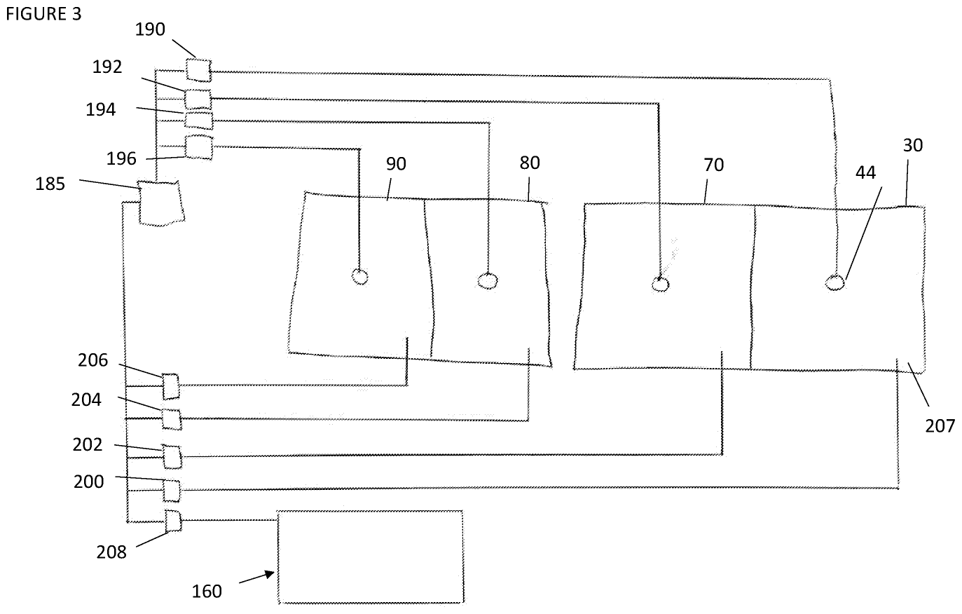

FIG. 3 is a schematic plan view of another part of the animal or human cremation apparatus of FIG. 1, in particular a plurality of tanks arranged in liquid communication with an effluent tank 160, and

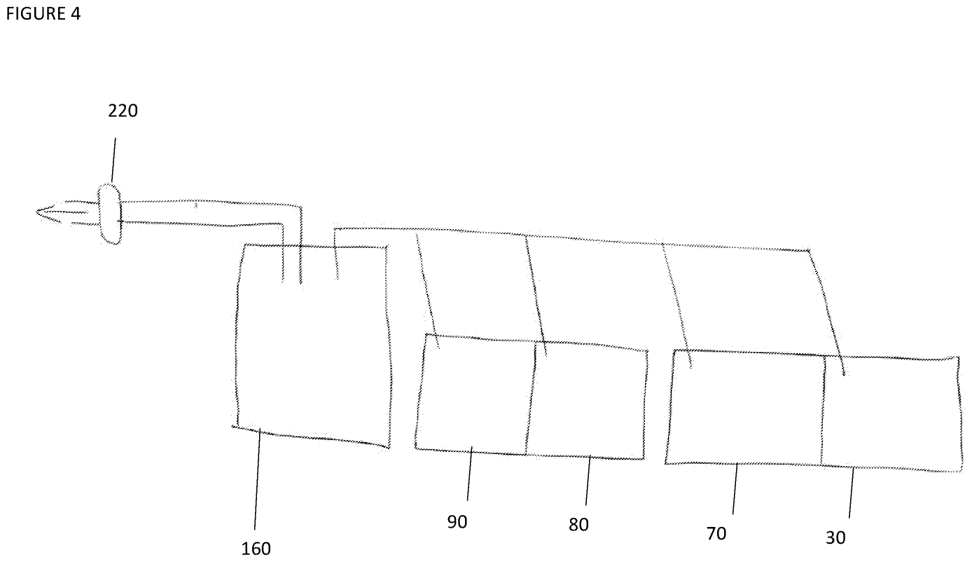

FIG. 4 is a schematic side view of another part of the animal or human cremation apparatus of FIG. 1, in particular a plurality of tanks arranged in gas communication with an effluent tank and a fan.

Referring to FIG. 1, in an embodiment of the invention, an animal or human cremation apparatus comprises a first tank 30 and a second tank 70. In other embodiments of the invention, any number of a plurality of tanks can be employed.

A tank comprises a base 32, and side walls 34, 36. The tank 30 comprises a rear wall 38, and front wall that has been removed in the cutaway for clarity. Therefore, the tank 30 is of rectangular cross section from above. In the embodiment shown, a single tank 30 is conveniently 2.5 m×1.25 m×1.25 m, conferring a volumetric capacity of 3.91 m³. Other size tanks can be employed, typically between 0.005 m³ and 5 m³. The tank 30 is designed to be filled or part filled with liquid.

In an embodiment not shown for conciseness, the size of the tank 30 can be varied, depending on the size of the animal or human cadaver, by using one or more movable wall in the tank inwardly or outwardly, so as to enable to the volume of water required to cover the cadaver to be minimised, thereby enabling energy to be saved in heating the volume of water.

In the embodiment shown in FIG. 1, the tanks 30, 70 are adjacent each other, and share common wall 36, for benefits in the material requirements for its construction. But, in another preferred embodiment, the tanks 30, 70 are separate detached units. The tanks 30, 70 can be arranged in abutment with each other or slightly spaced apart. The tanks 30, 70 can be insulated, so as to reduce heat flow from a hot fluid in one tank to another tank with cold fluid.

The tank 30 comprises a false floor 40 made from stainless steel mesh. Heaters 42 are arranged between the base 32 of the tank 30 and the false floor 40.

The base 32 of the tank 30 comprises a drain point 44.

The tank 30 comprises adjustable levelling feet 46 arranged below the base 32 of the tank.

The animal or human cremation apparatus comprises a plurality of enclosure baskets to be arranged in the tanks 30, 70. One or more basket can be arranged in each tank. In the embodiment shown, one basket 48 is shown in tank 30.

The sides 50, 52 and base 54 of the basket 48 permit liquid to pass therethrough but not solid matter such as bones. The sides 50, 52 and base 54 of the basket 48 may conveniently be made of a rigid mesh material. The mesh defines openings of between 1 mm and 5 mm.

The basket 48 comprises restraint means 56 positioned between the sides 50, 52 of the basket 48 so as to restrain the cadaver 58 to stop them floating on the liquid surface. In one embodiment, restraint means 56 comprises one or a plurality of stainless steel rods arranged between the sides 50, 52 of the basket 48. In another embodiment, not shown for conciseness, the restraint means 56 can be movable, from a first configuration (in which the cadaver 58 is restrained in the basket) to a second configuration (in which the cadaver 58 is not restrained in the basket), and vice versa.

Referring to FIG. 2, the animal or human cremation apparatus comprises a fresh water supply 100, arranged in liquid communication with the first tank 30, the second tank 70, a third tank 80, and a fourth tank 90.

Each of the first tank 30, the second tank 70, the third tank 80, and the fourth tank 90 comprises a rinse device, but only a rinse device 110 of the first tank 30 is labelled so as to simplify FIG. 2 of the drawings.

The rinse device 110 is mounted to the lid of the first tank 30. It comprises a (stainless steel) horizontal inlet pipe 112 and a sprinkler 114 rotatably mounted on the inlet pipe 112, so as to rotate in the horizontal plane. The rotatable sprinkler 114 is in liquid communication with water in the fresh water supply 100. The sprinkler 114 conveniently comprises multiple sprinkler heads.

The animal or human cremation apparatus also comprises a fresh water pump 120, and a plurality of rinse liquid valves. In the embodiment shown, a first rinse liquid valve 130, a second rinse liquid valve 132, a third rinse liquid valve 134, and a fourth rinse liquid valve 136, are, respectively, arranged between the pump 120 and the rinse device 110 of the first tank 30, and the pump 120 and each of the rinse devices of the second tank 70, the third tank 80, and the fourth tank 90, so that liquid can be controllably pumped from the fresh water supply 100 to each of the rinse devices 110.

The animal or human cremation apparatus also comprises a plurality of fill valves. In the embodiment shown, a first fill valve 140 is arranged in a fill line between the pump 120 and (an upper part of) the first tank 30, and a second fill valve 142, a third fill valve 144, and a fourth fill valve 146, are arranged in fill lines between the pump 120 and, respectively, (an upper part of) each of the second tank 70, the third tank 80, and the fourth tank 90.

In an alternative embodiment of the invention, which is not shown for conciseness, the fill lines and fill valves 140, 142, 144, 146 can be omitted, and the rinse devices 110 can perform the additional function of filling the tanks.

Referring to FIG. 3, the animal or human cremation apparatus comprises an effluent tank 160, arranged in liquid communication with the drain point 44 of the first tank 30, and drain points of the second tank 70, a third tank 80, and a fourth tank 90. Only a drain point 44 of the first tank 30 is labelled so as to simplify FIG. 2 of the drawings.

The animal or human cremation apparatus also comprises a second (effluent) pump 185, and a plurality of drain point valves. In the embodiment shown, a first drain point valve 190, a second drain point valve 192, a third drain point valve 194, and a fourth drain point valve 196, are, respectively, arranged between the pump 185 and the drain point 44 of the first tank 30, and the pump 185 and the drain points of each of the second tank 70, the third tank 80, and the fourth tank 90.

The animal or human cremation apparatus also comprises a plurality of effluent return valves. In the embodiment shown, a first effluent return valve 200, a second effluent return valve 202, a third effluent return valve 204, and a fourth effluent return valve 206, are, respectively, arranged between the effluent pump 185 and an effluent return point 207 of the first tank 30, and the pump 120 and fill points of each of the second tank 70, the third tank 80, and the fourth tank 90, so as to return effluent from a first tank to a second tank.

The animal or human cremation apparatus also comprises a master effluent tank valve 208, arranged between the effluent tank 160 and each of the first tank 30, the second tank 70, the third tank 80, and the fourth tank 90. The first fill valve 200, the second fill valve 202, the third fill valve 204, and the fourth fill valve 206 and the pump 185 are operable so as to transfer effluent from one or more of the first tank 30, the second tank 70, the third tank 80, and the fourth tank 90 to the effluent tank 160.

Referring to FIG. 4, the animal or human cremation apparatus also comprises an effluent vapour removal fan 220, arranged in gas communication with an upper part of the effluent tank 160. In turn, the effluent tank 160 is arranged in gas communication with an upper part of each of the first tank 30, the second tank 70, the third tank 80, and the fourth tank 90, using conventional ducting means not shown more than schematically for conciseness. The individual feeds of pipeline that approach each of the tanks extend around 1.5 metres vertically from the tanks.

In use, the animal or human cremation process is as follows.

A deceased animal or human cadaver 58 is arranged in the basket 48, and the basket 48 is lowered into the tank 30. The upper end of the sides 50, 52 of the basket 48 enable a basket to be safely lowered into the tank 30. Only one basket 48 is shown in FIG. 1 for clarity in the drawing, but a plurality of baskets may be arranged in a tank 30.

Appropriate dry chemical for an alkaline hydrolysis process is dosed on top of cadaver(s).

The restraint means 56 is position across the upper sides 50, 52 of the basket 48 so as to hold the cadaver 58 fully submerged under water during the cremation process, and retain the cadaver 58 in the basket 48 during the cremation process, and later in the cremation process so as to retain the bones of the cadaver in the basket 48. In an alternative embodiment no shown for conciseness the retrain means can be mounted on the tank 30 itself, for example on an inner wall thereof.

Then, the lid of the tank 30 is closed and locked.

The first fill side valve 140 is operated so that a measured amount of water from the fresh water supply 100 is pumped by pump 120 to the tank 30. The predetermined amount of water and the dry chemical creates the appropriate chemical mix in the fluid for the ensuing cremation process.

The water level is shown by line W in FIG. 1.

The heaters 42 are started and operate for the required duration.

The boiling point of the fluid and chemical mixture in the tank 30 is about the same as atmospheric water boiling point, 100 degrees. Allowing the fluid to boil, in turn allows the fluid to move around the tank 30, and reduces the need to mechanically agitate the fluid, so that it mixes. Water bubbles and moves at temperatures as low as about 70 degrees, so the cremation process might in theory be operated as low as that so as to obtain the mixing benefits associated with the movement of the bubbling fluid. Processing time is reduced if the temperature is above about 98 degrees, and there is not much difference in processing time between about 98 degrees and 100 degrees. Vapour production is limited if the temperature is below about 100 degrees. As such, the optimum operating temperature of the fluid in the tank 30 is about 98 degrees to 100 degrees.

The effluent vapour removal fan 220 should be operated above about 70 degrees, so as to remove vapour from about the temperature at which it starts to be emitted from the liquid in the tank 30, and the fan should be operated so as to avoid pressure build up in the tank and consequential increase in temperature in the liquid in the tank 30.

The gaseous fluid in the tanks 30, 70 is designed to stay at slightly less than atmospheric pressure during the cremation process, e.g. between 0.8 bar and 1.1 bar, more optimally about 0.9 to 1.0 bar, due to the effect of the vapour removal fan 220.

The effluent vapour removal fan 220 is operated so as to avoid pressure build up of the fluid in the tank 30.

The restraint means 56 prevent the cadaver 58 from floating to the top of the basket 48 and to the surface of the liquid in the tank, which might limit the liquid coverage of parts of the cadaver. This feature obtains an improved processing time.

During the cremation process, due to the upward extent of the effluent vapour pipelines adjacent each of the tanks 30, 70, 80, 90, steam can condense in the pipelines adjacent the tanks, and any steam that condenses in the pipes as it cools will be fed by gravity directly back into the tanks 30, 70, 80, 90. The physical characteristics of the pipework and the delta in temperature between the steam and the cooler surrounding pipework may be varied accordingly so as to obtain the desired condensing.

Remaining steam vapour that has not been condensed in the effluent vapour pipelines is carried along the high temperature pipe and fed into the effluent storage tank 160, where it condenses into liquid. The dry, hot air that remains of the effluent vapour in the effluent storage tank 160 is carried from the effluent storage tank 160 out of the building, via the fan 220, and discharged to atmosphere.

All ductwork is sealed, so the effluent vapour removal fan 220 can be operated so as to create a pressure slightly below atmospheric pressure, which in turn creates a negative pressure in the pipeline between the effluent tank 160 and the effluent vapour removal fan 220, and in the pipeline between the tank 160 and each of the first tank 30, the second tank 70, the third tank 80, and the fourth tank 90, thereby actively causing steam and other gases from the tanks 30, 70, 80, 90 to be discharged through an external vent to atmosphere.

Foam on the liquid surface in the tank 30 is difficult/slower to wash away before the tank 30 can be reused. Also, foam can effectively lift the highly alkaline effluent in the tank 30 up and into contact with the seals, creating a health and safety issue if any of the effluent foam breaches the seals under pressure and becomes uncontained.

The above mentioned solution is observed to work exceptionally well and maintains a slightly lower than atmospheric pressure in the tanks 30, 70, 80, 90 at all times, almost eliminating foaming on the liquid surface.

Yet the slightly lower than atmospheric pressure in the tanks 30, 70, 80, 90 allows the natural (aggressive) bubbling of boiling liquid to act as a very effective fluid circulation mechanism that keeps liquid actively flowing over the biological remains to speed up decomposition. Using this system eliminates the requirement to have an active recirculation system, as detailed in the prior art.

Once the process concludes, the effluent is pumped away (either to another tank, for example second tank 40, for reuse or to the effluent storage tank 160 if it is no longer needed).

The valves 190, 192, 194, 196, 200, 202, 204, 206, and 208, are employed to direct effluent fluid from one location to another. If it is desired to transfer effluent fluid from the first tank 30 to the second tank 70, the operator simply closes all drain point valves 192, 194, 196 other than the drain point valve 190 of the first tank 30 which is opened, and closes all return valves 200, 204, 206 other than the return valve 202 of the second tank 70 which is opened. Then upon starting the pump 185, fluid flows from the first tank 30, out of the drain 44 in the direction of the arrow in FIG. 1, to the second tank 70.

Once the effluent has been evacuated, the first rinse liquid valve 130 is operated so as to activate the rinse device 110, to rinse the (non liquid such as bone) remains of the cadaver 58 in the basket 48.

In particular, the rotatable sprinkler 114 is arranged to douse everything in the tank 30 below in water at high pressure. The fresh water pump pressure is sufficiently high to perform the function of removing the post cremation fluid from the bones.

The rinse water is also pumped away to the same location as the earlier used effluent, utilizing the valves 190, 192, 194, 196, 200, 202, 204, 206, and 208.

The tank 30 is now safe and is opened.

The upper end of the sides 50, 52 of the basket 48 enable a basket to be safely manually raised out of the tank 30.

The restraint means 56 is moved so as to allow the bones from the cadaver 58 to be removed from the basket 48.

The bones from the cadaver can be removed and further processed into ash for return to the owner of the deceased animal or human.

It should be appreciated by the reader that liquid can be moved from any tank 30, 70, 80, 90 to any other tank 30, 70, 80, 90 easily.

Effluent is not disposed of in the public drain.

The animal or human cremation apparatus comprises temperature sensors in each of the tanks 30, 70, 80, 90. The temperature sensors are preferably arranged under the water line, and more preferably between the heating elements 42.

The animal or human cremation apparatus may comprise an electronic controller, not shown for conciseness. The cremation process may be controlled by an electronic controller. For example, the temperature and pressure may be programmed and/or controlled.

1-27. (canceled)

28. An animal or human water cremation apparatus, comprising:
a first receptacle designed to contain a liquid suitable for carrying out alkaline hydrolysis on an animal or human cadaver;
a second receptacle designed to contain a liquid suitable for carrying out alkaline hydrolysis on an animal or human cadaver;
the first receptacle having a fresh water inlet;
the second receptacle having a fresh water inlet;
heating means for heating the liquid in the first receptacle and the second receptacle to form a waste liquid;
means for separating the waste liquid in the first receptacle from the second receptacle; and
means for transferring the waste liquid from the first receptacle to the second receptacle.

29. The animal or human cremation apparatus according to claim 28, further comprising means for transferring the waste liquid from the second receptacle to the first receptacle.

30. The animal or human cremation apparatus according to claim 28, further comprising an effluent storage tank, wherein the effluent storage tank is operable to receive the waste liquid from either the first or second receptacles.

31. The animal or human cremation apparatus according to claim 28, further comprising a plurality of fill valves, wherein a fill valve is associated with either the first or second receptacles, so as to control filling of the associated receptacle.

32. The animal or human cremation apparatus according to claim 28, further comprising a pump arranged to pump freshwater filling liquid to either the first or second receptacles.

33. The animal or human cremation apparatus according to claim 28, wherein either the first or second receptacles comprises a rinse device arranged in liquid communication with water in the fresh water supply to rinse bones of the animal or human cadaver after alkaline hydrolysis has been carried out on an animal or human cadaver.

34. The animal or human cremation apparatus according to claim 33, wherein the rinse device comprises a rotatable sprinkler.

35. The animal or human cremation apparatus according to claim 33, wherein the rinse device is designed to rinse an entire content of either the first or second receptacles.

36. The animal or human cremation apparatus according to claim 32, further comprising a second pump arranged to pump waste liquid from the first receptacle to the second receptacle, or from the second receptacle to the first receptacle.

37. The animal or human cremation apparatus according to claim 28, further comprising a plurality of effluent return valves, an effluent return valve being associated with either the first or second receptacles, so as to control effluent return to the associated receptacle.

38. The animal or human cremation apparatus according to claim 30, further comprising an effluent tank valve, arranged to control flow between the effluent storage tank and either the first or second receptacles.

39. The animal or human cremation apparatus according to claim 30, further comprising an effluent vapor removal fan, arranged in gas communication with either the first or second receptacles.

40. The animal or human cremation apparatus according to claim 39, wherein the effluent vapor removal fan is arranged in gas communication with either the first or second receptacles via ducting, wherein the ducting is arranged so that steam can condense in the pipelines adjacent to either the first or second receptacles, wherein liquid formed by condensed steam in the pipes is fed by gravity directly back into either the first or second receptacles.

41. The animal or human cremation apparatus according to claim 39, wherein the effluent vapor removal fan is arranged in gas communication with either the first or second receptacles via the effluent storage tank.

42. An animal or human water cremation apparatus, comprising:
a receptacle designed to contain a liquid suitable for carrying out alkaline hydrolysis on an animal or human cadaver;
wherein the receptacle is designed to heat the liquid to, and maintain the liquid at, a temperature of about 70 degrees Celsius to about 100 degrees Celsius, and is designed to maintain the gaseous liquid at a pressure of between about 0.9 to 1.0 bar.

43. The animal or human water cremation apparatus according to claim 42, wherein the receptacle is designed to heat the liquid to, and maintain the liquid at, a temperature of about 98 degrees to about 100 degrees Celsius.

44. The animal or human cremation apparatus according to claim 42, further comprising an effluent vapor removal fan arranged in gas communication with the receptacle.

45. The animal or human cremation apparatus according to claim 44, wherein the effluent vapor removal fan is arranged in gas communication with the receptacle via ducting, wherein the ducting is arranged so that steam can condense in the pipelines adjacent to the receptacle, and the apparatus is designed so that liquid formed by condensed steam in the pipes is fed by gravity directly back into the receptacle.

46. The animal or human cremation apparatus according to claim 42, further comprising a plurality of receptacles, wherein the plurality of receptacles comprises a first alkaline hydrolysis receptacle designed to contain a liquid suitable for carrying out alkaline hydrolysis on an animal or human cadaver, and a second alkaline hydrolysis receptacle designed to contain a liquid suitable for carrying out alkaline hydrolysis on an animal or human cadaver, the first receptacle having a fresh water inlet, and the second receptacle having a fresh water inlet, and heating means for heating liquid in the first receptacle and the second receptacle, means for separating hot waste liquid in the first receptacle from the second receptacle, and means for transferring hot waste liquid from the first receptacle to the second receptacle.

47. The animal or human cremation apparatus according to claim 46, further comprising a removable basket having walls made of mesh.

48. The animal or human cremation apparatus according to claim 47, further comprising a restraint means arrangeable across an upper part of the basket.

49. A method of cremating animals or humans, comprising the step of using an animal or human cremation apparatus in accordance with claim 28.
